(19) United States
(12) Patent Application Publication
(10) Pub. No.: US 2026/0034348 A1
(43) Pub. Date: Feb. 5, 2026

(54) Wound Management Systems

(71) Applicant: Vomaris Innovations, Inc., Fountain Hills, AZ (US)

(72) Inventor: Mary Maijer, Tempe, AZ (US)

(21) Appl. No.: 18/698,595

(22) PCT Filed: Oct. 4, 2022
(86) PCT No.: PCT/US2022/077507

§ 371 Date: Apr. 4, 2024

Related U.S. Application Data
(60) Provisional application No. 63/252,258, filed on Oct. 5, 2021.

(51) Int. Cl. A61N 1/04; A61F 13/0206

(52) U.S. Cl. CPC A61N 1/0468; A61F 13/0206; A61N 1/0496

(57) ABSTRACT

The instant disclosure provides systems and devices for wound management.

### CROSS-REFERENCE TO RELATED APPLICATION

This application claims the benefit of the filing date of U.S. Appl. No. 63/252,258, filed Oct. 5, 2021, which is incorporated herein by reference in its entirety.

### FIELD

The instant Specification discloses improved wound management systems.

### BACKGROUND

A Medical Adhesive-Related Skin Injury (MARSI) is a skin condition caused by repeated application or removal of medical adhesive products or devices such as tapes, wound dressings, stoma products, electrodes, medication patches and wound closure strips. The injury can occur when the attachment between the skin and an adhesive is stronger than that between individual cells, causing either the epidermal layers to separate or the epidermis to detach completely from the dermis (mechanical trauma.

Typically, medical adhesive-related skin injuries occur following removal of an adhesive medical device from patients whose skin is fragile or losing integrity. However, anyone who has a medical adhesive applied to them is at risk, with the risk increasing with the frequency of exposure (application and removal). The injury can cause significant pain and distress, and contribute to:
a. Longer hospital stays;
b. Longer treatment times in the community;
c. Increased health professional time;
d. Increased dressing usage;
e. Increased morbidity;
f. Delayed wound healing;
g. Scarring;
h. Increased risk of complications including infection.

The condition is especially problematic during the healing process as it can cause pain, damage, and infection, which increases recovery time as well as delaying healing and increasing the risk of scarring.

Typically, the injury is one of:
a. Skin Tear—Wound caused by shear, friction and/or blunt force resulting in separation of skin layers. Can be partial- or full-thickness.
b. Skin Stripping—Removal of one or more layers of the stratum corneum following adhesive removal. Lesions are frequently shallow and irregular in shape. Skin may appear shiny.
c. Tension Injury or Blister—Separation of the epidermis from the dermis caused by shear force as a result of distension of skin under an unyielding tape or dressing.
d. Maceration—Changes in the skin resulting from moisture being trapped against the skin for a prolonged period. Skin appears wrinkled and white/gray in color.

The incidence of MARSI varies dramatically, but appears to be closely related to a patient's age and condition. For example, Wounds International reported that 1.5 million skin tears occur each year in elderly patients in long-term senior care facilities in the US each year. The problem is also associated with very young patients, such as in neonatal intensive care, and 8% of hospitalized infants and children were found to have tape-related skin stripping.

Further, certain types of wounds and conditions can prove especially problematic for treatment. For example, treatment of wounds that are actively draining can be particularly challenging.

Therefore, improved systems and methods are desirable.

### SUMMARY

Disclosed and claimed herein are systems, devices, and methods for managing wounds and speeding recovery while reducing the risk of both viral and bacterial infection. Disclosed systems provide alternatives to industry-standard dressings, thus enabling the user to better monitor and treat wounds, while providing an improved patient experience.

Disclosed embodiments comprise dressing systems comprising a “flap,” “window,” or “door” that can open to provide access to the wound or a wound dressing, then close to provide a secure, clean, environment. In embodiments, the dressing system or a part or parts thereof is clear or transparent. For example, in embodiments, the adhesive, cover substrate, or window can be clear or transparent.

Disclosed embodiments comprise multi-layer systems wherein an electrode substrate contacts the treatment area, and the electrode substrate is covered with an absorbent substrate, all of which is covered by “cover” substrate comprising a “flap,” “window,” or “door” that can open to provide access to a wound or wound dressing, then close to provide a secure, clean, environment.

Disclosed embodiments comprise “smart” wound dressings that can monitor wound characteristics and healing.

In embodiments the systems, devices, and methods include absorbent substrates such as fabrics, for example dressings. In embodiments, disclosed absorbent dressings can comprise one or more biocompatible electrodes configured to generate at least one of a low level electric field (LLEF) or low level electric current (LLEC). Embodiments disclosed herein can produce a uniform current or field density.

Embodiments are directed toward methods for treating a patient with the disclosed systems and devices. Further embodiments are directed toward methods for manufacturing disclosed system and devices.

Patients with certain slow-healing conditions often require multiple wound dressing changes a day. Due to lesion location and size and frequency of change, dressings are hard to place, hard to change, and require leak-resistant high absorbency.

Only one dedicated wound dressing product that addresses these issues is currently available, but it requires a special undergarment (HidraWear) and does not provide antimicrobial protection

Disclosed embodiments better-address this issue by minimizing skin irritants (such as frequent adhesive changes to keep dressings in place) as well as providing antimicrobial protection.

Disclosed embodiments comprise devices and methods for treatment or avoidance of skin conditions. For example, hidradenitis suppurativa is a chronic skin condition featuring lumps in places such as the armpits or groin. Skin lesions develop as a result of inflammation and infection of sweat glands, and can develop in association with areas where skin dressings are applied and removed. This condition results in pea- to marble-sized lumps under the skin that can be painful and tend to enlarge and drain pus. Medications, corticosteroid injections, and sometimes surgery can help manage symptoms, however the effectiveness of current treatment methods is limited.

### BRIEF DESCRIPTION OF THE DRAWINGS

FIG. 1 shows a disclosed “window” dressing embodiment. The clear dressing allows visibility of the treatment area, while the “window” provides access so that a dressing may be changed multiple times without subjecting the patient to repeated removal/reattachment of adhesive.

FIG. 2 shows a disclosed “window” dressing embodiment.

FIG. 3 shows is a detailed plan view of an embodiment disclosed herein.

FIG. 4 is a detailed plan view of a pattern of applied electrical conductors in accordance with an embodiment disclosed herein.

FIG. 5 is a detailed plan view of an embodiment disclosed herein which includes fine lines of conductive metal solution connecting electrodes.

FIG. 6 is a detailed plan view of an embodiment having a line pattern and dot pattern.

FIG. 7 is a detailed plan view of an embodiment having two line patterns.

FIG. 8A is an Energy Dispersive X-ray Spectroscopy (EDS) analysis of Ag/Zn BED (“bioelectric device” refers to an embodiment as disclosed herein).
a. Scanning Electron Microscope (SEM) image;
b. Light Microscope Image;
c. Closer view of a golden dot and a grey dot in B respectively.
d. Closer view of a golden dot and a grey dot in B respectively.
e. EDS element map of zinc;
f. EDS element map of silver;
g. EDS element map of oxygen;
h. EDS element map of carbon.

Scale bar A-B, E-H: 1 mm; C-D: 250 μm

FIG. 8B and FIG. 8C—Absorbance measurement on treating planktonic PA01 culture with placebo, Ag/Zn BED and placebo+Ag dressing; and CFU measurement.

FIG. 8D—Zone of inhibition with placebo, Ag/Zn BED and placebo+Ag dressing.

FIG. 9 depicts Scanning Electron Microscope (SEM) images of in-vitro Pseudomonas aeruginosa PA01 biofilm treated with placebo, an embodiment disclosed herein (“BED”), and placebo+Ag dressing. The BED treated biofilm shows a dramatic decrease in bacteria number.

FIG. 10 shows extracellular polysaccharide staining (EPS).

FIG. 11 shows live/dead staining. The green fluorescence indicates live PA01 bacteria while the red fluorescence indicates dead bacteria.

FIG. 12 shows PAO1 staining.

FIG. 13 depicts real-time PCR to assess quorum sensing gene expression.

FIG. 14 shows electron paramagnetic (EPR) spectra using DEPMPO (a phosphorylated derivative of the widely used DMPO spin trap). Spin adduct generation upon exposure to disclosed embodiments for 40 minutes in PBS.

FIG. 15 depicts real-time PCR performed to assess mex gene expression upon treatment with Ag/Zn BED and 10 mM DTT.

FIG. 16 shows the antiviral effect of a disclosed embodiment.

FIG. 17 shows the antiviral effect of a disclosed embodiment.

FIG. 18 shows an overall wound-dressing concept of disclosed embodiments.

FIG. 19 shows an overall wound-dressing concept of disclosed embodiments. The “drop” indicates the addition of a conductive liquid to the device.

FIG. 20 shows an overall wound-dressing concept of disclosed embodiments.

### DETAILED DESCRIPTION

Skin surrounding wounds is subject to varying degrees of traumatic insult, such as the repeated application and removal of wound dressings/tapes or cleansing procedures. Cutaneous reactions to bandage adhesives is a well-recognized complication. It becomes especially problematic for patients with wounds requiring frequent dressing changes; chronic wounds such as Venous leg ulcers (VLUs), Hidradenitis suppurativa (HS) lesions, infected wounds, and many others are such wounds. Epithelial lifting, blistering, tissue cuts and tears may occur, compounding wound healing problems and further increasing risk of infection.

For patients with draining wounds, such as HS patients, dressings must be changed multiple times each day. Adhesives, such as those used to secure typical dressings, can be very irritating to the skin if they are removed frequently, and each time they are removed they “strip” some of the epithelial layer. Overtime, this can be painful and damage healthy skin barrier integrity. In addition, the wound perimeter skin area may already be very sensitive due to wound inflammation. As such, current practices are not an optimal solution for securing dressings in place and conducting repeated dressing changes, for example in cases of actively draining wounds.

In contrast, disclosed and claimed herein are systems, devices, and methods for managing wounds. For example, disclosed systems and devices provide alternatives to industry-standard dressings, enabling the user to better monitor and treat wounds, while providing an improved patient experience.

Disclosed embodiments comprise dressing systems comprising a perimeter adhesive portion that secures the system to the patient, and a “flap” or “window” or “door” within the perimeter that can open to provide access to the wound or wound dressing, then close to provide a secure, clean, environment.

Disclosed embodiments provide an antimicrobial wound dressing that can be changed as frequently as desired without the need to remove the perimeter adhesive every time the dressing is changed, because the system provides repeated wound access without breaking the adhesive seal.

### Definitions

“Activation agent” as used herein means a composition useful for maintaining a moist environment within and about the skin, such as in a treatment location. Activation agents can be in the form of gels or liquids. Activation agents can be conductive. Activation gels can also be antibacterial. In one embodiment, an activation agent can be a liquid such as perspiration or topical substance such as petroleum jelly (for example with a conductive component added).

“Affixing” as used herein can mean contacting a patient or tissue with a device or system disclosed herein. In embodiments “affixing” can comprise the use of straps, elastic, etc.

“Antimicrobial agent” as used herein refers to an agent that kills or inhibits the growth of microorganisms. One type of antimicrobial agent can be an antibacterial agent. “Antibacterial agent” or “antibacterial” as used herein refers to an agent that interferes with the growth and reproduction of bacteria. Antibacterial agents are used to disinfect surfaces and eliminate potentially harmful bacteria. Unlike antibiotics, they are not used as medicines for humans or animals, but are found in products such as soaps, detergents, health and skincare products and household cleaners.

“Applied” or “apply” as used herein refers to contacting a surface with a conductive material, for example printing, painting, or spraying a conductive ink on a surface. Alternatively, “applying” can mean contacting a patient or tissue or organism with a device or system disclosed herein.

“Conductive material” as used herein refers to an object or type of material which permits the flow of electric charges in one or more directions. Conductive materials can comprise solids such as metals or carbon, or liquids such as conductive metal solutions and conductive gels.

“Discontinuous region” as used herein refers to a “void” in a material such as a hole, slot, slit, or the like. The term can mean any void in the material, though typically the void is of a regular shape. Avoid in the material can be entirely within the perimeter of a material or it can extend to the perimeter of a material. The discontinuous region can be linear, such as a slot to provide a flap.

“Dots” as used herein refers to discrete deposits of dissimilar reservoirs that can function as at least one battery cell. The term can refer to a deposit of any suitable size or shape, such as squares, circles, triangles, lines, etc. The term can be used synonymously with, microcells, microspheres, etc. “Microspheres” refers to small spherical particles, with diameters in the micrometer range (typically 1 μm to 3000 μm (3 mm)). Microspheres are sometimes referred to as microparticles. Microspheres can be manufactured from various natural and synthetic materials. The term can be used synonymously with, microballoons, beads, particles, etc.

“Electrode” refers to similar or dissimilar conductive materials. In embodiments utilizing an external power source the electrodes can comprise similar conductive materials. In embodiments that do not use an external power source, the electrodes can comprise dissimilar conductive materials that can define an anode and a cathode.

“Expandable” as used herein refers to the ability to stretch while retaining structural integrity and not tearing. The term can refer to solid regions as well as discontinuous or void regions, solid regions as well as void regions can stretch or expand.

“Matrix” or “matrices” or “array” or “arrays” as used herein refer to a pattern or patterns, such as those formed by electrodes on a surface, such as a fabric or a fiber, or the like. Matrices can also comprise a pattern or patterns within a solid or liquid material or a three dimensional object. Matrices can be designed to vary the electric field or electric current or microcurrent generated.

“Stretchable” as used herein refers to the ability of embodiments that stretch without losing their structural integrity. That is, embodiments can stretch to accommodate irregular skin surfaces or surfaces wherein one portion of the surface can move relative to another portion.

“Treatment” as used herein can include the use of disclosed embodiments on an injury, for example a wound such as an actively draining wound, by contacting a disclosed device or system to the area to be treated.

### Wound Management Systems

Through the use of disclosed embodiments, dressings can be changed multiple times through a given time period without having to detach and reapply adhesive around the treatment site, as the cover substrate remains attached through multiple openings/closings of the window. Further embodiments comprise, for example within the perimeter of the covering substrate, a “window” or “door” that can reversibly open and close. When open, access to the treatment site is provided, and dressings can be changed or checked. When closed, the wound site is protected.

Disclosed embodiments can be employed for any type of condition where frequent dressing changes are required and skin condition surrounding the perimeter of the wound area is a concern. For example, elderly patients with fragile skin, trauma wounds that require multiple days of dressing changes, infected and heavily draining wounds, and venous stasis ulcers can all be treated successfully with disclosed systems and methods without detachment/reattachment of the cover substrate.

### Cover Substrates

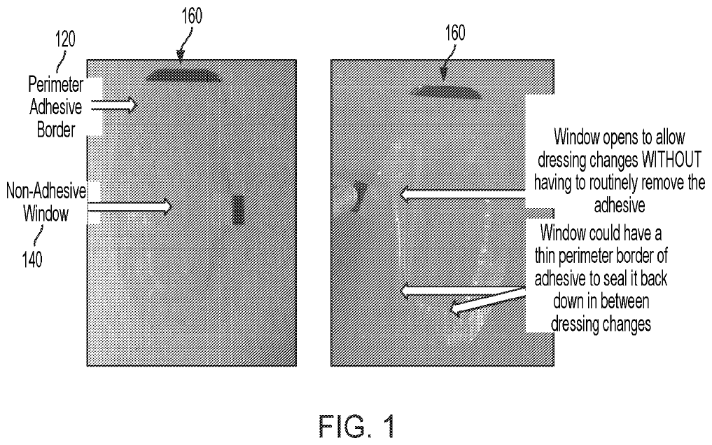

In embodiments, disclosed wound management systems and dressings comprise a cover substrate comprising, for example, an adhesive perimeter as in FIG. 1 at 120 surrounding a “window” or “flap” 140.

In embodiments, the entire perimeter of the covering substrate comprises an adhesive. In embodiments, the adhesive is present on only a part or parts of the perimeter of the cover substrate. For example, in embodiments, the portion of the perimeter comprising the adhesive can be 10%, 20%, 30%, 40%, 50%, 60%, 70%, 80%, 90%, or 100% of the perimeter.

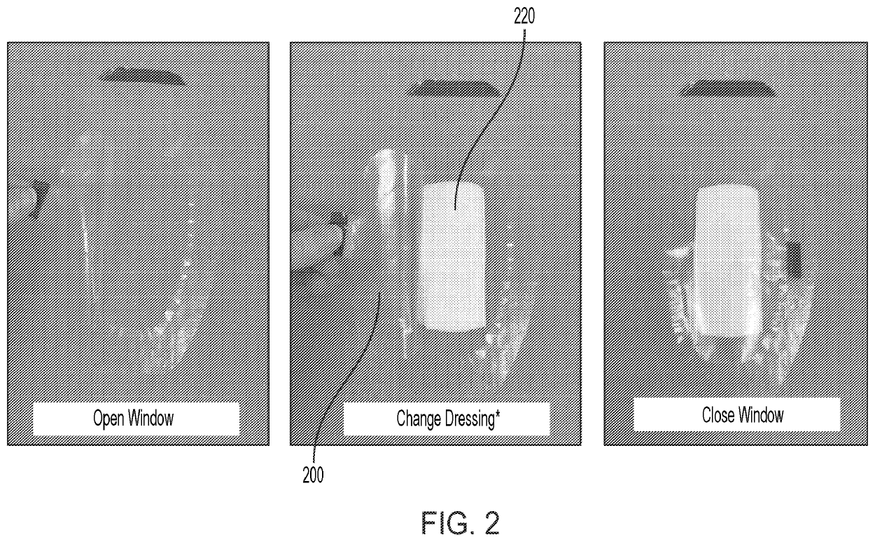

In embodiments, the “window” or “flap” can be opened to access and change an absorbent substrate, for example a dressing 220, as seen in FIG. 2. In embodiments, a clear substrate cover comprises an adhesive perimeter surrounding a “window” 200 that can be opened to access and change a dressing.

In FIG. 1, pull-tabs 160 along the perimeter of the covering substrate and the window provide means for opening the dressing and removing and changing a wound-contacting substrate, for example an absorbent substrate. The window is opened to access the treatment site. In embodiments, the window comprises an adhesive. In embodiments, the adhesive is present on only a part or parts of the perimeter of the window. For example, in embodiments, the portion of the window perimeter comprising the adhesive can be 10%, 20%, 30%, 40%, 50%, 60%, 70%, 80%, 90%, or 100% of the perimeter. FIG. 2 illustrates the opening and closing of the window. In embodiments, adhesive on the window, for example the window perimeter, reversibly attaches to the cover substrate, thus avoiding the wound area.

In embodiments, the cover substrate or a part or parts thereof can be, for example, clear, transparent, or opaque.

In embodiments, the system further comprises an absorbent substrate to accommodate the fluid draining from the treatment area. In embodiments, the absorbent substrate is smaller than the device such that it can fit within the perimeter of the device.

In embodiments, the system further comprises a substrate comprising electrodes. In embodiments, the substrate comprising electrodes is smaller than the device such that it can fit within the perimeter of the device.

### Absorbent Substrates

Disclosed systems can comprise absorbent substrates. For example, absorbent substrates can comprise any suitable wound-contacting material that provides an absorbent effect, such as, for example, cotton, fabrics, gauze, polymeric materials, and the like. In embodiments, the absorbent substrate is sized to fit within the perimeter of the cover substrate.

Disclosed absorbent substrates can also comprise electrodes or dots or microcells. Each electrode or dot or microcell can be or comprise a conductive metal. In embodiments, the electrodes or microcells can comprise any electrically-conductive material, for example, an electrically conductive hydrogel, metals, electrolytes, superconductors, semiconductors, plasmas, and nonmetallic conductors such as graphite and conductive polymers. Electrically conductive metals can comprise silver, copper, gold, aluminum, molybdenum, zinc, lithium, tungsten, brass, carbon, nickel, iron, palladium, platinum, tin, bronze, carbon steel, lead, titanium, stainless steel, mercury, Fe/Cr alloys, and the like. The electrode can be solid, coated or plated with a different metal such as aluminum, gold, platinum or silver. Disclosed substrates can produce a low-level electric field (LLEF), a low-level electric current (LLEC), or both.

Alternatively, the absorbent substrate and the substrate comprising the electrodes can be separate layers.

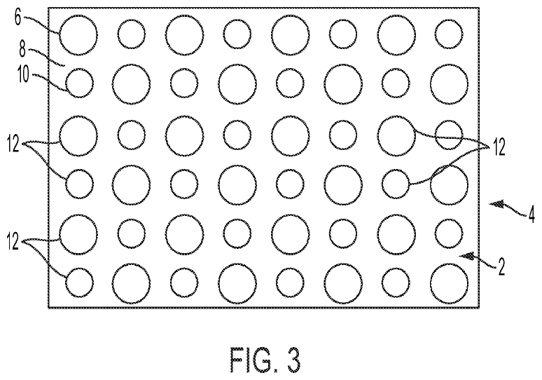

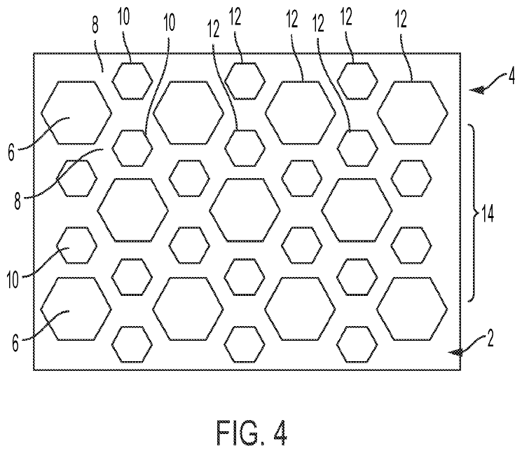

In certain embodiments, reservoir or electrode geometry can comprise circles, polygons, lines, zigzags, ovals, stars, or any suitable variety of shapes, such as in FIG. 3 and FIG. 4. This provides the ability to design/customize surface electric field shapes as well as depth of penetration.

Reservoir or electrode or dot sizes and concentrations can vary, as these variations can allow for changes in the properties of the electric field created by embodiments of the invention. Certain embodiments provide an electric field at about, for example, 0.5-5.0 V at the device surface under normal tissue loads with resistance of 100 to 100K ohms.

In embodiments devices disclosed herein can produce an electric field, an electric current, or both, wherein the field, current, or both can be of varying size, strength, density, shape, or duration in different areas of the embodiment. In embodiments, by micro-sizing the electrodes or reservoirs, the shapes of the electric field, electric current, or both can be customized, increasing or decreasing very localized watt densities and allowing for the design of patterns of electrodes or reservoirs wherein the amount of electric field over a tissue can be designed or produced or adjusted based upon feedback from the tissue or upon an algorithm within sensors operably connected to the embodiment and a control module. The electric field, electric current, or both can be stronger in one zone and weaker in another.

Dissimilar metals used to make a LLEC or LLEF system disclosed herein can comprise, for example, silver and zinc.

In another embodiment, substrates can be formed, coated, and plated by printing. In embodiments, printing devices can be used to produce LLEC or LLEF systems disclosed herein. For example, inkjet or “3D” printers can be used to produce embodiments. In certain embodiments the binders or inks used to produce LLEC or LLEF systems disclosed herein can comprise, for example, poly cellulose inks, poly acrylic inks, poly urethane inks, silicone inks, and the like. Other materials, such as silicon, can be added to enhance, for example, scar reduction. Such materials can also be added to the spaces between reservoirs.

A dot pattern of masses like the alternating round dots of FIG. 3 can be preferred when applying conductive material onto a flexible material, such as those used for an absorbent substrate, as the dots won't significantly affect the flexibility of the material. To maximize the density of electrical current over a primary surface the pattern of FIG. 4 can be used. The first electrode 6 in FIG. 4 is a large hexagonally shaped dot, and the second electrode 10 is a pair of smaller hexagonally shaped dots that are spaced from each other. The spacing 8 that is between the first electrode 6 and the second electrode 10 maintains a relatively consistent distance between adjacent sides of the designs. Numerous repetitions 12 of the designs result in a pattern 14 that can be described as at least one of the first design being surrounded by six hexagonally shaped dots of the second design.

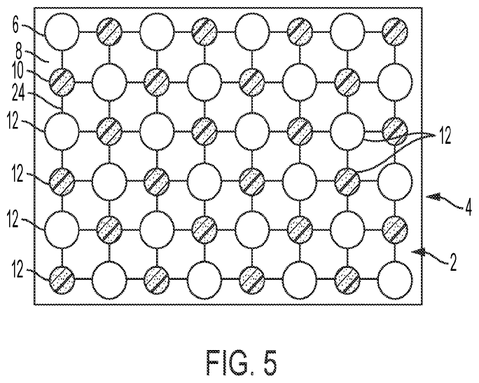

FIG. 5 shows an additional feature, which can be added between designs, that can initiate the flow of current in a poor electrolytic solution. A fine line 24 is printed using one of the conductive metal solutions along a current path of each voltaic cell. The fine line will initially have a direct reaction but will be depleted until the distance between the electrodes increases to where maximum voltage is realized. The initial current produced is intended to help control edema so that the LLEC system will be effective. If the electrolytic solution is highly conductive when the system is initially applied the fine line can be quickly depleted and the device will function as though the fine line had never existed.

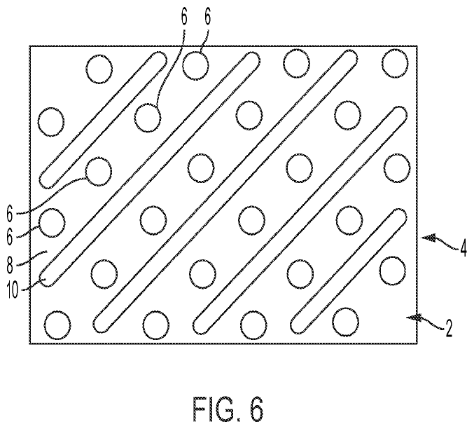

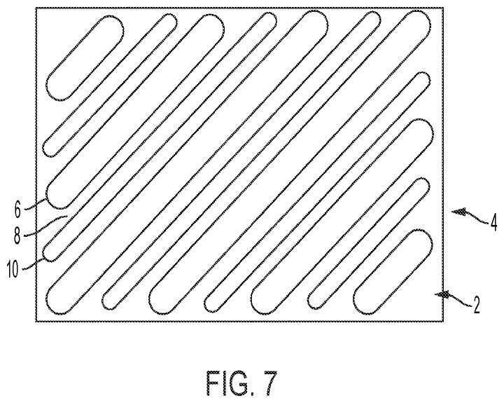

FIGS. 6 and 7 show alternative patterns that use at least one line design. The first electrode 6 of FIG. 6 is a round dot similar to the first design used in FIG. 1. The second electrode 10 of FIG. 6 is a line. When the designs are repeated, they define a pattern of parallel lines that are separated by numerous spaced dots. FIG. 7 uses only line designs. The first electrode 6 can be thicker or wider than the second electrode 10 if the oxidation-reduction reaction requires more metal from the first conductive element (mixed into the first designs conductive metal solution) than the second conductive element (mixed into the second design's conductive metal solution). The lines can be dashed.

In embodiments, systems and devices disclosed herein can produce a low level electric current of between for example about 1 and about 200 micro-amperes, between about 10 and about 190 micro-amperes, between about 20 and about 180 micro-amperes, between about 30 and about 170 micro-amperes, between about 40 and about 160 micro-amperes, between about 50 and about 150 micro-amperes, between about 60 and about 140 micro-amperes, between about 70 and about 130 micro-amperes, between about 80 and about 120 micro-amperes, between about 90 and about 100 micro-amperes, between about 100 and about 150 micro-amperes, between about 150 and about 200 micro-amperes, between about 200 and about 250 micro-amperes, between about 250 and about 300 micro-amperes, between about 300 and about 350 micro-amperes, between about 350 and about 400 micro-amperes, between about 400 and about 450 micro-amperes, between about 450 and about 500 micro-amperes, between about 500 and about 550 micro-amperes, between about 550 and about 600 micro-amperes, between about 600 and about 650 micro-amperes, between about 650 and about 700 micro-amperes, between about 700 and about 750 micro-amperes, between about 750 and about 800 micro-amperes, between about 800 and about 850 micro-amperes, between about 850 and about 900 micro-amperes, between about 900 and about 950 micro-amperes, between about 950 and about 1000 micro-amperes (1 milli-amp [mA]), between about 1.0 and about 1.1 mA, between about 1.1 and about 1.2 mA, between about 1.2 and about 1.3 mA, between about 1.3 and about 1.4 mA, between about 1.4 and about 1.5 mA, between about 1.5 and about 1.6 mA, between about 1.6 and about 1.7 mA, between about 1.7 and about 1.8 mA, between about 1.8 and about 1.9 mA, between about 1.9 and about 2.0 mA, between about 2.0 and about 2.1 mA, between about 2.1 and about 2.2 mA, between about 2.2 and about 2.3 mA, between about 2.3 and about 2.4 mA, between about 2.4 and about 2.5 mA, between about 2.5 and about 2.6 mA, between about 2.6 and about 2.7 mA, between about 2.7 and about 2.8 mA, between about 2.8 and about 2.9 mA, between about 2.9 and about 3.0 mA, between about 3.0 and about 3.1 mA, between about 3.1 and about 3.2 mA, between about 3.2 and about 3.3 mA, between about 3.3 and about 3.4 mA, between about 3.4 and about 3.5 mA, between about 3.5 and about 3.6 mA, between about 3.6 and about 3.7 mA, between about 3.7 and about 3.8 mA, between about 3.8 and about 3.9 mA, between about 3.9 and about 4.0 mA, between about 4.0 and about 4.1 mA, between about 4.1 and about 4.2 mA, between about 4.2 and about 4.3 mA, between about 4.3 and about 4.4 mA, between about 4.4 and about 4.5 mA, between about 4.5 and about 5.0 mA, between about 5.0 and about 5.5 mA, between about 5.5 and about 6.0 mA, between about 6.0 and about 6.5 mA, between about 6.5 and about 7.0 mA, between about 7.5 and about 8.0 mA, between about 8.0 and about 8.5 mA, between about 8.5 and about 9.0 mA, between about 9.0 and about 9.5 mA, between about 9.5 and about 10.0 mA, between about 10.0 and about 10.5 mA, between about 10.5 and about 11.0 mA, between about 11.0 and about 11.5 mA, between about 11.5 and about 12.0 mA, between about 12.0 and about 12.5 mA, between about 12.5 and about 13.0 mA, between about 13.0 and about 13.5 mA, between about 13.5 and about 14.0 mA, between about 14.0 and about 14.5 mA, between about 14.5 and about 15.0 mA, or the like.

In embodiments, systems and devices disclosed herein can produce a low level electric current of between for example about 1 and about 400 micro-amperes, between about 20 and about 380 micro-amperes, between about 40 and about 360 micro-amperes, between about 60 and about 340 micro-amperes, between about 80 and about 320 micro-amperes, between about 100 and about 3000 micro-amperes, between about 120 and about 280 micro-amperes, between about 140 and about 260 micro-amperes, between about 160 and about 240 micro-amperes, between about 180 and about 220 micro-amperes, or the like.

In embodiments, systems and devices disclosed herein can produce a low level electric current of about 10 micro-amperes, about 20 micro-amperes, about 30 micro-amperes, about 40 micro-amperes, about 50 micro-amperes, about 60 micro-amperes, about 70 micro-amperes, about 80 micro-amperes, about 90 micro-amperes, about 100 micro-amperes, about 110 micro-amperes, about 120 micro-amperes, about 130 micro-amperes, about 140 micro-amperes, about 150 micro-amperes, about 160 micro-amperes, about 170 micro-amperes, about 180 micro-amperes, about 190 micro-amperes, about 200 micro-amperes, about 210 micro-amperes, about 220 micro-amperes, about 240 micro-amperes, about 260 micro-amperes, about 280 micro-amperes, about 300 micro-amperes, about 320 micro-amperes, about 340 micro-amperes, about 360 micro-amperes, about 380 micro-amperes, about 400 micro-amperes, about 450 micro-amperes, about 500 micro-amperes, about 550 micro-amperes, about 600 micro-amperes, about 650 micro-amperes, about 700 micro-amperes, about 750 micro-amperes, about 800 micro-amperes, about 850 micro-amperes, about 900 micro-amperes, about 950 micro-amperes, about 1 milli-ampere, or the like.

Because the spontaneous oxidation-reduction reaction of silver and zinc uses a ratio of approximately two silver to one zinc, in embodiments the silver design can contain about twice as much mass as the zinc design in an embodiment. Spacing between the closest conductive materials can be, for example, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, 30 μm, 31 μm, 32 μm, 33 μm, 34 μm, 35 μm, 36 μm, 37 μm, 38 μm, 39 μm, 40 μm, 41 μm, 42 μm, 43 μm, 44 μm, 45 μm, 46 μm, 47 μm, 48 μm, 49 μm, 50 μm, 51 μm, 52 μm, 53 μm, 54 μm, 55 μm, 56 μm, 57 μm, 58 μm, 59 μm, 60 μm, 61 μm, 62 μm, 63 μm, 64 μm, 65 μm, 66 μm, 67 μm, 68 μm, 69 μm, 70 μm, 71 μm, 72 μm, 73 μm, 74 μm, 75 μm, 76 μm, 77 μm, 78 μm, 79 μm, 80 μm, 81 μm, 82 μm, 83 μm, 84 μm, 85 μm, 86 μm, 87 μm, 88 μm, 89 μm, 90 μm, 91 μm, 92 μm, 93 μm, 94 μm, 95 μm, 96 μm, 97 μm, 98 μm, 99 μm, 0.1 mm, or 0.2 mm, 0.3 mm, 0.4 mm, 0.5 mm, 0.6 mm, 0.7 mm, 0.8 mm, 0.9 mm, 1 mm, 1.1 mm, 1.2 mm, 1.3 mm, 1.4 mm, 1.5 mm, 1.6 mm, 1.7 mm, 1.8 mm, 1.9 mm, 2 mm, 2.1 mm, 2.2 mm, 2.3 mm, 2.4 mm, 2.5 mm, 2.6 mm, 2.7 mm, 2.8 mm, 2.9 mm, 3 mm, 3.1 mm, 3.2 mm, 3.3 mm, 3.4 mm, 3.5 mm, 3.6 mm, 3.7 mm, 3.8 mm, 3.9 mm, 4 mm, 4.1 mm, 4.2 mm, 4.3 mm, 4.4 mm, 4.5 mm, 4.6 mm, 4.7 mm, 4.8 mm, 4.9 mm, 5 mm, 5.1 mm, 5.2 mm, 5.3 mm, 5.4 mm, 5.5 mm, 5.6 mm, 5.7 mm, 5.8 mm, 5.9 mm, 6 mm, or the like.

Disclosures absorbent substrates of the present Specification can comprise LLEC or LLEF systems comprising a hydrophilic polymer base and a first electrode design formed from a first conductive liquid that comprises a mixture of a polymer and a first element, the first conductive liquid being applied into a position of contact with the primary surface, the first element comprising a metal species, and the first electrode design comprising at least one dot or reservoir, wherein selective ones of the at least one dot or reservoir have approximately a 1.5 μm+/−1 μm mean diameter; a second electrode design formed from a second conductive liquid that comprises a mixture of a polymer and a second element, the second element comprising a different metal species than the first element, the second conductive liquid being printed into a position of contact with the primary surface, and the second electrode design comprising at least one other dot or reservoir, wherein selective ones of the at least one other dot or reservoir have approximately a 2 μm+/−2 μm mean diameter; a spacing on the primary surface that is between the first electrode design and the second electrode design such that the first electrode design does not physically contact the second electrode design, wherein the spacing is approximately 1.5 μm+/−1 μm, and at least one repetition of the first electrode design and the second electrode design, the at least one repetition of the first electrode design being substantially adjacent the second electrode design, wherein the at least one repetition of the first electrode design and the second electrode design, in conjunction with the spacing between the first electrode design and the second electrode design, defines at least one pattern of at least one voltaic cell for spontaneously generating at least one electrical current when introduced to an electrolytic solution. Therefore, electrodes, dots or reservoirs can have a mean diameter of, for example, about 0.2 μm, 0.3 μm, 0.4 μm, 0.5 μm, 0.6 μm, 0.7 μm, 0.8 μm, 0.9 μm, 1.0 μm, 1.1 μm, 1.2 μm, 1.3 μm, 1.4 μm, 1.5 μm, 1.6 μm, 1.7 μm, 1.8 μm, 1.9 μm, 2.0 μm, 2.1 μm, 2.2 μm, 2.3 μm, 2.4 μm, 2.5 μm, 2.6 μm, 2.7 μm, 2.8 μm, 2.9 μm, 3.0 μm, 3.1 μm, 3.2 μm, 3.3 μm, 3.4 μm, 3.5 μm, 3.6 μm, 3.7 μm, 3.8 μm, 3.9 μm, 4.0 μm, 4.1 μm, 4.2 μm, 4.3 μm, 4.4 μm, 4.5 μm, 4.6 μm, 4.7 μm, 4.8 μm, 4.9 μm, 5.0 μm, or the like not exceeding 1 mm.

In various embodiments the difference of the standard potentials of the first and second reservoirs or electrodes or dots can be in a range from about 0.05 V to approximately 5.0 V. For example, the standard potential can be, for example, about 0.05 V, 0.06 V, 0.07 V, 0.08 V, 0.09 V, 0.1 V, 0.2 V, 0.3 V, 0.4 V, 0.5 V, 0.6 V, 0.7 V, 0.8 V, 0.9 V, 1.0 V, 1.1 V, 1.2 V, 1.3 V, 1.4 V, 1.5 V, 1.6 V, 1.7 V, 1.8 V, 1.9 V, 2.0 V, 2.1 V, 2.2 V, 2.3 V, 2.4 V, 2.5 V, 2.6 V, 2.7 V, 2.8 V, 2.9 V, 3.0 V, 3.1 V, 3.2 V, 3.3 V, 3.4 V, 3.5 V, 3.6 V, 3.7 V, 3.8 V, 3.9 V, 4.0 V, 4.1 V, 4.2 V, 4.3 V, 4.4 V, 4.5 V, 4.6 V, 4.7 V, 4.8 V, 4.9 V, 5.0 V, or the like.

Embodiments can comprise coatings on the surface of the substrate, such as, for example, over or between the electrodes or cells or an excipient or activation agent suspended within the coating. Coatings can comprise, for example, silicone, and electrolytic mixture, hypoallergenic agents, drugs, biologics, stem cells, skin substitutes, cosmetic products, combinations, or the like. Drugs suitable for use with embodiments of the invention comprise analgesics, antibiotics, anti-inflammatories, or the like.

In further embodiments the device or system can comprise a conductive material, for example a wire to electrically link the device with other components, such as monitoring equipment or a power source.

In embodiments the device can be a “smart” device, for example wirelessly linked to monitoring or data collection equipment, for example linked via Bluetooth to a cell phone or computer that collects data from the device. In certain embodiments the device can comprise data collection means, such as temperature, pH, pressure, or conductivity data collection means. In certain embodiments, disclosed devices and systems can comprise data collection means, such as temperature, pH, pressure, or conductivity data collection means. Embodiments can comprise a display, for example to visually present, for example, the temperature, pH, pressure, or conductivity data to a user.

In embodiments the system comprises a component such as an adhesive to maintain or help maintain its position. The adhesive component can be covered with a protective layer that is removed to expose the adhesive at the time of use. In embodiments the adhesive can comprise, for example, sealants, such as hypoallergenic sealants, gecko sealants, mussel sealants, waterproof sealants such as epoxies, and the like. Straps can comprise Velcro or similar materials to aid in maintaining the position of the device.

In certain embodiments, a substrate comprising an array can comprise one layer of a composite dressing, for example a composite garment or fabric comprising any or all of the substrate, an adhesive layer, an expandable absorbent layer, and a stretchable, expandable film layer. The expandable absorbent layer can absorb excess fluid from the substrate and expand away from the treatment area, thus preventing oversaturation of the treatment area with resultant maceration and increased infection risk. The stretchable, expandable film layer can stretch to accommodate a larger foam volume as the foam absorbs liquid. This aspect reduces shear forces on the skin. Additionally, the vertically-expanding foam and film allows the dressing to absorb more volume of fluid in a smaller contact area.

In embodiments, the LLEC or LLEF system can comprise instructions or directions on how to place the system to maximize its performance. Embodiments comprise a kit comprising an LLEC or LLEF system and directions for its use.

In certain embodiments dissimilar metals can be used to create an electric field with a desired voltage. In certain embodiments the pattern of reservoirs can control the watt density and shape of the electric field.

Certain embodiments can utilize a power source to create the electric current, such as a battery or a micro-battery. The power source can be any energy source capable of generating a current in the LLEC system and can comprise, for example, AC power, DC power, radio frequencies (RF) such as pulsed RF, induction, ultrasound, and the like.

Similarly, electrodes or reservoirs or dots can adhere or bond to a substrate through use of a biocompatible binder. Conductive metal solutions can comprise a binder mixed with a conductive element. The resulting conductive metal solution can be used with an application method such as screen printing to apply the electrodes to the primary surface in predetermined patterns. Once the conductive metal solution dries and/or cures, the patterns of spaced electrodes can substantially maintain their relative position, even on a flexible material such as that used for a LLEC or LLEF system. The conductive metal solution can be allowed to dry before being applied to a surface.

The binder can comprise any biocompatible liquid material that can be mixed with a conductive element (preferably metallic crystals of silver or zinc) to create a conductive solution which can be applied as a thin coating to a microsphere. One suitable binder is a solvent reducible polymer, such as the polyacrylic non-toxic silk-screen ink manufactured by COLORCON® Inc., a division of Berwind Pharmaceutical Services, Inc. (see COLORCON® NO-TOX® product line, part number NT28). In an embodiment the binder is mixed with high purity (at least 99.999%) metallic silver crystals to make the silver conductive solution. The silver crystals, which can be made by grinding silver into a powder, are preferably smaller than 100 microns in size or about as fine as flour. In an embodiment, the size of the crystals is about 325 mesh, which is typically about 40 microns in size or a little smaller. The binder is separately mixed with high purity (at least 99.99%, in an embodiment) metallic zinc powder which has also preferably been sifted through standard 325 mesh screen, to make the zinc conductive solution. For better quality control and more consistent results, most of the crystals used should be larger than 325 mesh and smaller than 200 mesh.

Other powders of metal can be used to make other conductive metal solutions in the same way as described in other embodiments.

For LLEC or LLEF systems comprising a pliable substrate it can be desired to decrease the percentage of metal down to 5 percent or less, or to use a binder that causes the crystals to be more deeply embedded, so that the primary surface will be antimicrobial for a very long period of time and will not wear prematurely. Other binders can dissolve or otherwise break down faster or slower than a polyacrylic ink, so adjustments can be made to achieve the desired rate of spontaneous reactions from the voltaic cells.

In certain embodiments, the system can be shaped to fit a particular region of the body such as an arm, leg, ankle, chest, decubitus wound, or diabetic ulcer.

In embodiments, the width and depth of the various areas of the electrode can be designed to produce a particular electric field, or, when both electrodes are in contact with a conductive material, a particular electric current. For example, the width of the various areas of the electrode can be, for example, 0.1 mm, 0.2 mm, 0.3 mm, 0.4 mm, 0.5 mm, 0.6 mm, 0.7 mm, 0.8 mm, 0.9 mm, 1 mm, 1.1 mm, 1.2 mm, 1.3 mm, 1.4 mm, 1.5 mm, 1.6 mm, 1.7 mm, 1.8 mm, 1.9 mm, 2 mm, 2.1 mm, 2.2 mm, 2.3 mm, 2.4 mm, 2.5 mm, 2.6 mm, 2.7 mm, 2.8 mm, 2.9 mm, 3 mm, or the like.

The shortest distance between the two electrodes in an embodiment can be, for example, 0.1 mm, 0.2 mm, 0.3 mm, 0.4 mm, 0.5 mm, 0.6 mm, 0.7 mm, 0.8 mm, 0.9 mm, 1 mm, 1.1 mm, 1.2 mm, 1.3 mm, 1.4 mm, 1.5 mm, 1.6 mm, 1.7 mm, 1.8 mm, 1.9 mm, 2 mm, 2.1 mm, 2.2 mm, 2.3 mm, 2.4 mm, 2.5 mm, 2.6 mm, 2.7 mm, 2.8 mm, 2.9 mm, 3 mm, or the like.

### Methods of Treatment

Disclosed methods of treatment can comprise treatment of:
a. Skin tears;
b. Erosion or stripping of the skin;
c. Blisters or tension injuries;
d. Dermatitis;
e. Skin softening, wrinkling or breakdown due to moisture becoming trapped under an adhesive (maceration);
f. Inflammation or infection of hair follicles (folliculitis), which can occur if the moist and warm environment between the skin and adhesive tape or dressing attracts microbes, which then proliferate;
g. Abnormal reddening of the skin (erythema);
h. Hidradenitis suppurativa.

Disclosed embodiments comprise treatment of surgical incisions, infected wounds, diabetic foot ulcers, pressure ulcers, venous stasis ulcers, and the like.

Disclosed embodiments comprise treatment of wounds, for example an actively draining wound. For example, treatment of wounds can comprise applying a disclosed device or system to a wound. Embodiments disclosed herein relating to tissue treatment can also comprise selecting a patient or tissue in need of, or that could benefit by, treatment with a disclosed system or device.

Disclosed embodiments can comprise treatment of surgical wounds. In embodiments, the surgical wound can result from dental surgery.

In embodiments, methods can further comprise a tissue assessment, wherein characteristics are evaluated, such as:
a. Skin temperature;
b. Skin color;
c. Skin moisture level;
d. Skin turgor (fullness and elasticity);
e. Skin fragility; and
f. Skin integrity.

In embodiments, methods for treating or dressing a wound comprises the step of topically administering an additional material on the wound surface or upon the matrix of biocompatible microcells. These additional materials can comprise, for example, activation gels, rhPDGF (REGRANEX®), Vibronectin:IGF complexes, CELLSPRAY®, RECELL®, INTEGRA® dermal regeneration template, BIOMEND®, INFUSE®, ALLODERM®, CYMETRA®, SEPRAPACK®, SEPRAMESH®, SKINTEMP®, MEDFIL®, COSMODERM®, COSMOPLAST®, OP-1®, ISOLAGEN®, CARTICEL®, APLIGRAF®, DERMAGRAFT®, TRANSCYTE®, ORCEL®, EPICEL®, and the like. In embodiments the activation gel can be, for example, TEGADERM® 91110 by 3M, Mölnlycke Normlgel 0.9% Sodium chloride, HISPAGEL®, LUBRIGEL®, or other compositions useful for maintaining a moist environment about the wound or useful for healing a wound via another mechanism.

Aspects of the present specification provide, in part, methods of reducing a symptom associated with a wound. In an aspect of this embodiment the symptom reduced is edema, hyperemia, erythema, bruising, tenderness, stiffness, swollenness, fever, a chill, a breathing problem, fluid retention, a blood clot, a loss of appetite, an increased heart rate, a formation of granulomas, fibrinous, pus, or non-viscous serous fluid, a formation of an ulcer, or pain.

### EXAMPLES

The following non-limiting examples are provided for illustrative purposes only in order to facilitate a more complete understanding of representative embodiments. These examples should not be construed to limit any of the embodiments described in the present specification.

### Example 1

### Cell Migration Assay

The in vitro “scratch” assay is an easy, low-cost and well-developed method to measure cell migration in vitro. The basic steps involve creating a scratch in a cell monolayer, capturing images at the beginning and at regular intervals during cell migration to close the scratch, and comparing the images to quantify the migration rate of the cells. Compared to other methods, the in vitro scratch assay is particularly suitable for studies on the effects of cell-matrix and cell-cell interactions on cell migration, mimic cell migration during wound healing in vivo and are compatible with imaging of live cells during migration to monitor intracellular events if desired. In addition to monitoring migration of homogenous cell populations, this method has also been adopted to measure migration of individual cells in the leading edge of the scratch.

Human keratinocytes were plated under plated under placebo or a LLEC system. Cells were also plated under silver-only or zinc-only dressings. After 24 hours, the scratch assay was performed. Cells plated under the LLEC system displayed increased migration into the “scratched” area as compared to any of the zinc, silver, or placebo dressings. After 9 hours, the cells plated under the LLEC system had almost “closed” the scratch. This demonstrates the importance of electrical activity to cell migration and infiltration.

In addition to the scratch test, genetic expression was tested. Increased insulin growth factor (IGF)-1 R phosphorylation was demonstrated by the cells plated under the LLEC system as compared to cells plated under insulin growth factor alone.

Integrin accumulation also affects cell migration. An increase in integrin accumulation was achieved with the LLEC system. Integrin is necessary for cell migration, and is found on the leading edge of migrating cell.

Thus, the tested LLEC system enhanced cellular migration and IGF-1 R/integrin involvement. This involvement demonstrates the effect that the LLEC system had upon cell receptors involved with the wound healing process.

### Example 2

### Wound Care Study

The medical histories of patients who received “standard-of-care” wound treatment (“SOC”; n=20), or treatment with a LLEC device as disclosed herein (n=18), were reviewed. The wound care device used in the present study consisted of a discrete matrix of silver and zinc dots. A sustained voltage of approximately 0.8 V was generated between the dots. The electric field generated at the device surface was measured to be 0.2-1.0 V, 10-50 μA.

Wounds were assessed until closed or healed. The number of days to wound closure and the rate of wound volume reduction were compared. Patients treated with LLEC received one application of the device each week, or more frequently in the presence of excessive wound exudate, in conjunction with appropriate wound care management. The LLEC was kept moist by saturating with normal saline or conductive hydrogel. Adjunctive therapies (such as negative pressure wound therapy [NPWT], etc.) were administered with SOC or with the use of LLEC unless contraindicated. The SOC group received the standard of care appropriate to the wound, for example antimicrobial dressings, barrier creams, alginates, silver dressings, absorptive foam dressings, hydrogel, enzymatic debridement ointment, NPWT, etc. Etiology-specific care was administered on a case-by-case basis. Dressings were applied at weekly intervals or more. The SOC and LLEC groups did not differ significantly in gender, age, wound types or the length, width, and area of their wounds.

Wound dimensions were recorded at the beginning of the treatment, as well as interim and final patient visits. Wound dimensions, including length (L), width (W) and depth (D) were measured, with depth measured at the deepest point. Wound closure progression was also documented through digital photography. Determining the area of the wound was performed using the length and width measurements of the wound surface area.

Closure was defined as 100% epithelialization with visible effacement of the wound. Wounds were assessed 1 week post-closure to ensure continued progress toward healing during its maturation and remodeling phase.

Wound types included in this study were diverse in etiology and dimensions, thus the time to heal for wounds was distributed over a wide range (9-124 days for SOC, and 3-44 days for the LLEC group). Additionally, the patients often had multiple co-morbidities, comprising diabetes, renal disease, and hypertension. The average number of days to wound closure was 36.25 (SD=28.89) for the SOC group and 19.78 (SD=14.45) for the LLEC group, p=0.036. On average, the wounds in the LLEC treatment group attained closure 45.43% earlier than those in the SOC group.

Based on the volume calculated, some wounds improved persistently while others first increased in size before improving. The SOC and the LLEC groups were compared to each other in terms of the number of instances when the dimensions of the patient wounds increased (i.e., wound treatment outcome degraded). In the SOC group, 10 wounds (50% for n=20) became larger during at least one measurement interval, whereas 3 wounds (16.7% for n=18) became larger in the LLEC group (p=0.018). Overall, wounds in both groups responded positively. Response to treatment was observed to be slower during the initial phase, but was observed to improve as time progressed.

The LLEC wound treatment group demonstrated on average a 45.4% faster closure rate as compared to the SOC group. Wounds receiving SOC were more likely to follow a “waxing-and-waning” progression in wound closure compared to wounds in the LLEC treatment group.

Compared to localized SOC treatments for wounds, the LLEC (1) reduces wound closure time, (2) has a steeper wound closure trajectory, and (3) has a more robust wound healing trend with lower incidence of increased wound dimensions during the course of healing.

### Example 3

### LLEC Influence on Human Keratinocyte Migration

An LLEC-generated electrical field was mapped, leading to the observation that LLEC generates hydrogen peroxide, known to drive redox signaling. LLEC-induced phosphorylation of redox-sensitive IGF-1 R was directly implicated in cell migration. The LLEC also increased keratinocyte mitochondrial membrane potential.

The LLEC was made of polyester printed with dissimilar elemental metals. It comprises alternating circular regions of silver and zinc dots, along with a proprietary, biocompatible binder added to lock the electrodes to the surface of a flexible substrate in a pattern of discrete reservoirs. When the LLEC contacts an aqueous solution, the silver positive electrode (cathode) is reduced while the zinc negative electrode (anode) is oxidized. The LLEC used herein consisted of metals placed in proximity of about 1 mm to each other thus forming a redox couple and generating an ideal potential on the order of 1 Volt. The calculated values of the electric field from the LLEC were consistent with the magnitudes that are typically applied (1-10 V/cm) in classical electrotaxis experiments, suggesting that cell migration observed with the bioelectric dressing is likely due to electrotaxis.

Measurement of the potential difference between adjacent zinc and silver dots when the LLEC is in contact with de-ionized water yielded a value of about 0.2 Volts. Though the potential difference between zinc and silver dots can be measured, non-intrusive measurement of the electric field arising from contact between the LLEC and liquid medium was difficult. Keratinocyte migration was accelerated by exposure to an Ag/Zn LLEC. Replacing the Ag/Zn redox couple with Ag or Zn alone did not reproduce the effect of keratinocyte acceleration.

Exposing keratinocytes to an LLEC for 24 h significantly increased green fluorescence in the dichlorofluorescein (DCF) assay indicating generation of reactive oxygen species under the effect of the LLEC. To determine whether H₂O₂ is generated specifically, keratinocytes were cultured with a LLEC or placebo for 24 h and then loaded with PF6-AM (Peroxyfluor-6 acetoxymethyl ester, an indicator of endogenous H₂O₂). Greater intracellular fluorescence was observed in the LLEC keratinocytes compared to the cells grown with placebo. Over-expression of catalase (an enzyme that breaks down H₂O₂) attenuated the increased migration triggered by the LLEC. Treating keratinocytes with N-Acetyl Cysteine (which blocks oxidant-induced signaling) also failed to reproduce the increased migration observed with LLEC. Thus, H₂O₂ signaling mediated the increase of keratinocyte migration under the effect of the electrical stimulus.

External electrical stimulus can up-regulate the TCA (tricarboxylic acid) cycle. The stimulated TCA cycle is then expected to generate more NADH and FADH2 to enter into the electron transport chain and elevate the mitochondrial membrane potential (Am). Fluorescent dyes JC-1 and TMRM were used to measure mitochondrial membrane potential. JC-1 is a lipophilic dye which produces a red fluorescence with high Am and green fluorescence when Am is low. TMRM produces a red fluorescence proportional to Am. Treatment of keratinocytes with LLEC for 24 h demonstrated significantly high red fluorescence with both JC-1 and TMRM, indicating an increase in mitochondrial membrane potential and energized mitochondria under the effect of the LLEC. As a potential consequence of a stimulated TCA cycle, available pyruvate (the primary substrate for the TCA cycle) is depleted resulting in an enhanced rate of glycolysis. This can lead to an increase in glucose uptake in order to push the glycolytic pathway forward. The rate of glucose uptake in HaCaT cells treated with LLEC was examined next. More than two fold enhancement of basal glucose uptake was observed after treatment with LLEC for 24 h as compared to placebo control.

Keratinocyte migration is known to involve phosphorylation of a number of receptor tyrosine kinases (RTKs). To determine which RTKs are activated as a result of LLEC, scratch assay was performed on keratinocytes treated with LLEC or placebo for 24 h. Samples were collected after 3 h and an antibody array that allows simultaneous assessment of the phosphorylation status of 42 RTKs was used to quantify RTK phosphorylation. It was determined that LLEC significantly induces IGF-1 R phosphorylation. Sandwich ELISA using an antibody against phospho-IGF-1 R and total IGF-1 R verified this determination. As observed with the RTK array screening, potent induction in phosphorylation of IGF-1 R was observed 3 h post scratch under the influence of LLEC. IGF-1 R inhibitor attenuated the increased keratinocyte migration observed with LLEC treatment.

MBB (monobromobimane) alkylates thiol groups, displacing the bromine and adding a fluoresce nt tag (lamda emission=478 nm). MCB (monochlorobimane) reacts with only low molecular weight thiols such as glutathione. Fluorescence emission from UV laser-excited keratinocytes loaded with either MBB or MCB was determined for 30 min. Mean fluorescence collected from 10,000 cells showed a significant shift of MBB fluorescence emission from cells. No significant change in MCB fluorescence was observed, indicating a change in total protein thiol but not glutathione. HaCaT cells were treated with LLEC for 24 h followed by a scratch assay. Integrin expression was observed by immuno-cytochemistry at different time points. Higher integrin expression was observed 6 h post scratch at the migrating edge.

Consistent with evidence that cell migration requires H₂O₂ sensing, we determined that by blocking H₂O₂ signaling by decomposition of H₂O₂ by catalase or ROS scavenger, N-acetyl cysteine, the increase in LLEC-driven cell migration is prevented. The observation that the LLEC increases H₂O₂ production is significant because in addition to cell migration, hydrogen peroxide generated in the wound margin tissue is required to recruit neutrophils and other leukocytes to the wound, regulates monocyte function, and VEGF signaling pathway and tissue vascularization. Therefore, external electrical stimulation can be used as an effective strategy to deliver low levels of hydrogen peroxide over time to mimic the environment of the healing wound and thus should help improve wound outcomes. Another phenomenon observed during re-epithelialization is increased expression of the integrin subunit alpha-v. There is evidence that integrin, a major extracellular matrix receptor, polarizes in response to applied ES and thus controls directional cell migration. It may be noted that there are a number of integrin subunits, however we chose integrin aV because of evidence of association of alpha-v integrin with IGF-1 R, modulation of IGF-1 receptor signaling, and of driving keratinocyte locomotion. Additionally, integrin alpha v has been reported to contain vicinal thiols that provide site for redox activation of function of these integrins and therefore the increase in protein thiols that we observe under the effect of ES may be the driving force behind increased integrin mediated cell migration. Other possible integrins which may be playing a role in LLEC-induced IGF-1 R mediated keratinocyte migration are a5 integrin and a6 integrin.

### Materials and Methods

Cell culture-Immortalized HaCaT human keratinocytes were grown in Dulbecco's low-glucose modified Eagle's medium (Life Technologies, Gaithersburg, MD, U.S.A.) supplemented with 10% fetal bovine serum, 100 U/ml penicillin, and 100 μg/ml streptomycin. The cells were maintained in a standard culture incubator with humidified air containing 5% CO₂ at 37° C.

Scratch assay—A cell migration assay was performed using culture inserts (IBIDI®, Verona, WI) according to the manufacturer's instructions. Cell migration was measured using time-lapse phase-contrast microscopy following withdrawal of the insert. Images were analyzed using the AxioVision Rel 4.8 software.

N-Acetyl Cysteine Treatment—Cells were pretreated with 5 mM of the thiol antioxidant N-acetylcysteine (Sigma) for 1 h before start of the scratch assay.

IGF-1 R inhibition—When applicable, cells were preincubated with 50 nM IGF-1 R inhibitor, picropodophyllin (Calbiochem, MA) just prior to the Scratch Assay.

Cellular H₂O₂ Analysis—To determine intracellular H₂O₂ levels, HaCaT cells were incubated with 5 pM PF6-AM in PBS for 20 min at room temperature. After loading, cells were washed twice to remove excess dye and visualized using a Zeiss Axiovert 200M microscope.

Catalase gene delivery—HaCaT cells were transfected with 2.3×107 pfu AdCatalase or with the empty vector as control in 750 μl of media. Subsequently, 750 μl of additional media was added 4 h later and the cells were incubated for 72 h.

RTK Phosphorylation Assay—Human Phospho-Receptor Tyrosine Kinase phosphorylation was measured using Phospho-RTK Array kit (R & D Systems).

ELISA—Phosphorylated and total IGF-1 R were measured using a DuoSet IC ELISA kit from R&D Systems.

Determination of Mitochondrial Membrane Potential—Mitochondrial membrane potential was measured in HaCaT cells exposed to the LLEC or placebo using TMRM or JC-1 (MitoProbe JC-1 Assay Kit for Flow Cytometry, Life Technologies), per manufacturer's instructions for flow cytometry.

Integrin alpha V Expression—Human HaCaT cells were grown under the MCD or placebo and harvested 6 h after removing the IBIDI® insert. Staining was done using antibody against integrin aV (Abeam, Cambridge, MA).

### Example 4

### Generation of Superoxide

A LLEC system was tested to determine the effects on superoxide levels which can activate signal pathways. LLEC system increased cellular protein sulfhydryl levels. Further, the LLEC system increased cellular glucose uptake in human keratinocytes. Increased glucose uptake can result in greater mitochondrial activity and thus increased glucose utilization, providing more energy for cellular migration and proliferation. This can “prime” the wound healing process before a surgical incision is made and thus speed incision healing.

### Example 5

### Effect on Propionibacterium acnes

### Bacterial Strains and Culture

The main bacterial strain used in this study is Propionibacterium acnes and multiple antibiotics-resistant P. acnes isolates are to be evaluated.

ATCC medium (7 Actinomyces broth) (BD) and/or ATCC medium (593 chopped meat medium) is used for culturing P. acnes under an anaerobic condition at 37° C. All experiments are performed under anaerobic conditions.

### Culture

LNA (Leeming-Notman agar) medium is prepared and cultured at 34° C. for 14 days.

### Planktonic Cells

P. acnes is a relatively slow-growing, typically aero-tolerant anaerobic, Gram-positive bacterium (rod). P. acnes is cultured under anaerobic condition to determine for efficacy of an embodiment disclosed herein (LLEC system). Overnight bacterial cultures are diluted with fresh culture medium supplemented with 0.1% sodium thioglycolate in PBS to 105 colony forming units (CFUs). Next, the bacterial suspensions (0.5 mL of about 105) are applied directly on LLEC system (2″×2″) and control fabrics in Petri-dishes under anaerobic conditions. After 0 h and 24 h post treatments at 37° C., portions of the sample fabrics are placed into anaerobic diluents and vigorously shaken by vortexing for 2 min. The suspensions are diluted serially and plated onto anaerobic plates under an anaerobic condition. After 24 h incubation, the surviving colonies are counted. The LLEC limits bacterial proliferation.

### Example 6

### Metallic Gel Solution and Single-Metal Substrate

This study demonstrated an alternative method of producing a Redox reaction voltage between two metals (such as zinc and silver), without having both metals embedded in the same substrate. By removing one of the metals from the substrate and mixing it with a conductive gel, the voltage potential was comparable to the voltage potential of both metals embedded in the substrate (PROCELLERA®).

Observed Voltage Potential

| | Zinc only | Silver only |
|---|---|---|
| | Substrate | Substrate |
| Silver Gel | ≤.75 V | n/a |
| Solution | | |
| Zinc Gel | n/a | ≤.85 V |
| Solution | | |
| Pure Gel | ≤.45 V | ≤.25 V |
| Solution | | |

*Gel used was sterile AquaSonic 100 by Parker Labs

### Example 7

### Modulation of Bacterial Gene Expression and Enzyme Activity

Treatment of biofilms presents a major challenge, because bacteria living within them enjoy increased protection against host immune responses and are markedly more tolerant to antibiotics. Bacteria residing within biofilms are encapsulated in an extracellular matrix, consisting of several components including polysaccharides, proteins and DNA which acts as a diffusion barrier between embedded bacteria and the environment thus retarding penetration of antibacterial agents. Additionally, due to limited nutrient accessibility, the biofilm-residing bacteria are in a physiological state of low metabolism and dormancy increasing their resistance towards antibiotic agents.

Chronic wounds present an increasing socio-economic problem and an estimated 1-2% of western population suffers from chronic ulcers and approximately 2-4% of the national healthcare budget in developed countries is spent on treatment and complications due to chronic wounds. The incidence of non-healing wounds is expected to rise as a natural consequence of longer lifespan and progressive changes in lifestyle like obesity, diabetes, and cardiovascular disease. Non-healing skin ulcers are often infected by biofilms. Multiple bacterial species reside in chronic wounds, with Pseudomonas aeruginosa, especially in larger wounds, being the most common. P. aeruginosa is suspected to delay healing of leg ulcers. Also, surgical success with split graft skin transplantation and overall healing rate of chronic venous ulcers is presumably reduced when there is clinical infection by P. aeruginosa.

P. aeruginosa biofilm is often associated with chronic wound infection. The BED (“BED” or “bioelectric device” or PROCELLERA® as disclosed herein) consists of a matrix of silver-zinc coupled biocompatible microcells, which in the presence of conductive wound exudate activates to generate an electric field (0.3-0.9V). Growth (measured as O.D and cfu) of pathogenic Pseudomonas aeruginosa strain PA01 in LB media was markedly arrested in the presence of the BED (p<0.05, n=4). PA01 biofilm was developed in vitro using a polycarbonate filter model. Grown overnight in LB medium at 37° C. bacteria were cultured on sterile polycarbonate membrane filters placed on LB agar plates and allowed to form a mature biofilm for 48 h. The biofilm was then exposed to BED or placebo for the following 24 h. Structural characterization using scanning electron microscopy demonstrated that the BED markedly disrupted biofilm integrity as compared to no significant effect observed using a commercial silver dressing commonly used for wound care. Staining of extracellular polymeric substance, PA01 staining, and a vital stain demonstrated a decrease in biofilm thickness and number of live bacterial cells in the presence of BED (n=4). BED repressed the expression of quorum sensing genes lasR and rhIR (p<0.05, n=3). BED was also found to generate micromolar amounts of superoxide (n=3), which are known reductants and repress genes of the redox sensing multidrug efflux system mexAB and mexEF (n=3, p<0.05). BED also down-regulated the activity of glycerol-3-phosphate dehydrogenase, an electric field sensitive enzyme responsible for bacterial respiration, glycolysis, and phospholipid biosynthesis (p<0.05, n=3).

### Materials and Methods

### In-Vitro Biofilm Model

PA01 biofilm was developed in vitro using a polycarbonate filter model. Cells were grown overnight in LB medium at 37° C. bacteria were cultured on sterile polycarbonate membrane filters placed on LB agar plates and allowed to form a mature biofilm for 48 h. The biofilm was then exposed to BED or placebo for the following 24 h.

### Energy Dispersive X-Ray Spectroscopy (EDS)

EDS elemental analysis of the Ag/ZN BED was performed in an environmental scanning electron microscope (ESEM, FEI XL-30) at 25 k V. A thin layer of carbon was evaporated onto the surface of the dressing to increase the conductivity.

### Scanning Electron Microscopy

Biofilm was grown on circular membranes and was then fixed in a 4% formaldehyde/2% glutaraldehyde solution for 48 hours at 4° C., washed with phosphate-buffered saline solution buffer, dehydrated in a graded ethanol series, critical point dried, and mounted on an aluminum stub. The samples were then sputter coated with platinum (Pt) and imaged with the SEM operating at 5 kV in the secondary electron mode (XL 30S; FEG, FEI Co., Hillsboro, OR).

### Live/Dead Staining

The LIVE/DEAD BacLight Bacterial Viability Kit for microscopy and quantitative assays was used to monitor the viability of bacterial populations. Cells with a compromised membrane that are considered to be dead or dying stain red, whereas cells with an intact membrane stain green.

### EPR Spectroscopy

EPR measurements were performed at room temperature using a Bruker ER 300 EPR spectrometer operating at X-band with a TM 110 cavity. The microwave frequency was measured with an EIP Model 575 source-locking microwave counter (EIP Microwave, Inc., San Jose, CA). The instrument settings used in the spin trapping experiments were as follows: modulation amplitude, 0.32 G; time constant, 0.16 s; scan time, 60 s; modulation frequency, 100 kHz; microwave power, 20 mW; microwave frequency, 9.76 GHz. The samples were placed in a quartz EPR flat cell, and spectra were recorded at ambient temperature (25° C.). Serial 1-min EPR acquisitions were performed. The components of the spectra were identified, simulated, and quantitated as reported. The double integrals of DEPMPO experimental spectra were compared with those of a 1 mM TEMPO sample measured under identical settings to estimate the concentration of superoxide adduct.

Quantification of mRNA and miRNA Expression

Total RNA, including the miRNA fraction, was isolated using Norgen RNA isolation kit, according to the manufacturer's protocol. Gene expression levels were quantified with real-time PCR system and SYBR Green (Applied Biosystems) and normalized to nadB and proC as housekeeping genes. Expression levels were quantified employing the 2 (−ΔΔct) relative quantification method.

### Glycerol-3-Phosphate Dehydrogenase Assay

The glycerol-3-phosphate dehydrogenase assay was performed using an assay kit from Biovision, Inc. following manufacturer's instructions. Briefly, cells (˜1×10⁶) were homogenized with 200 pi ice cold GPDH Assay buffer for 10 minutes on ice and the supernatant was used to measure O.D. and GPDH activity calculated from the results.

### Statistics

Control and treated samples were compared by paired t test. Student's t test was used for all other comparison of difference between means. P<0.05 was considered significant.

Ag/Zn BED Disrupts P. aeruginosa Biofilm

To validate the chemical composition of the dressing, we collected high resolution electron micrographs using an environmental scanning electron microscope. Our element maps indicate that silver particles are concentrated in the golden dots of the polyester cloth, while zinc particles are concentrated in the grey dots.

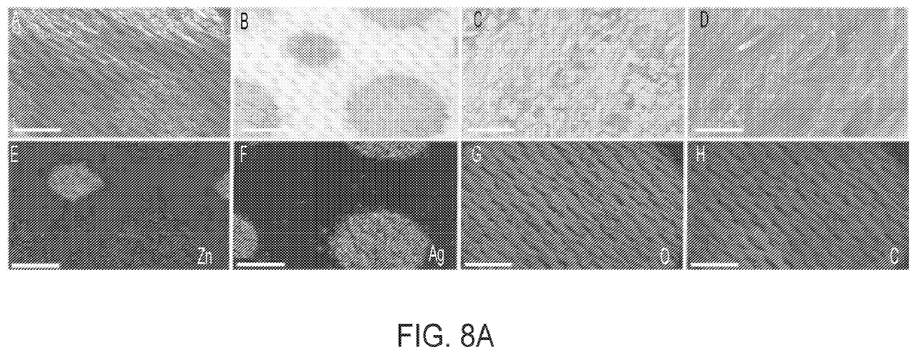

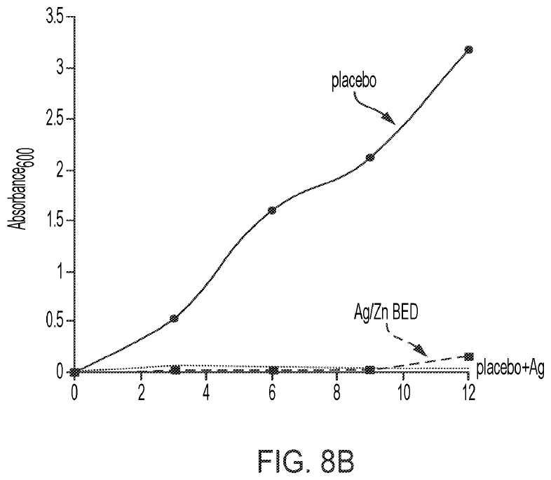

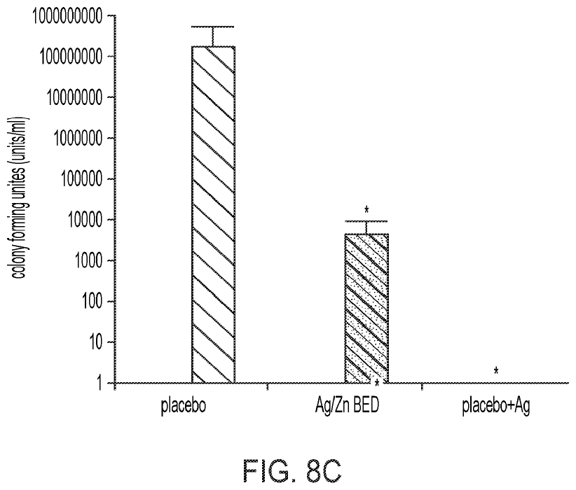

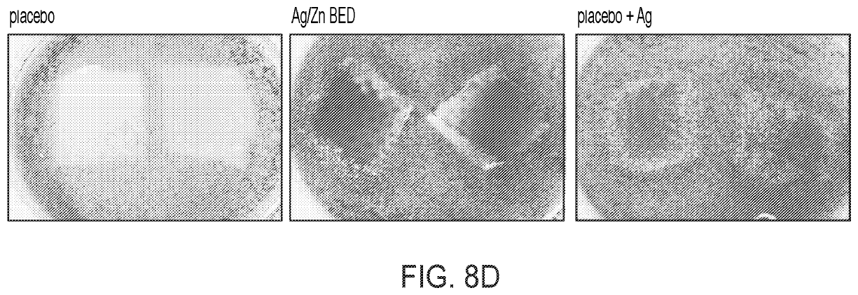

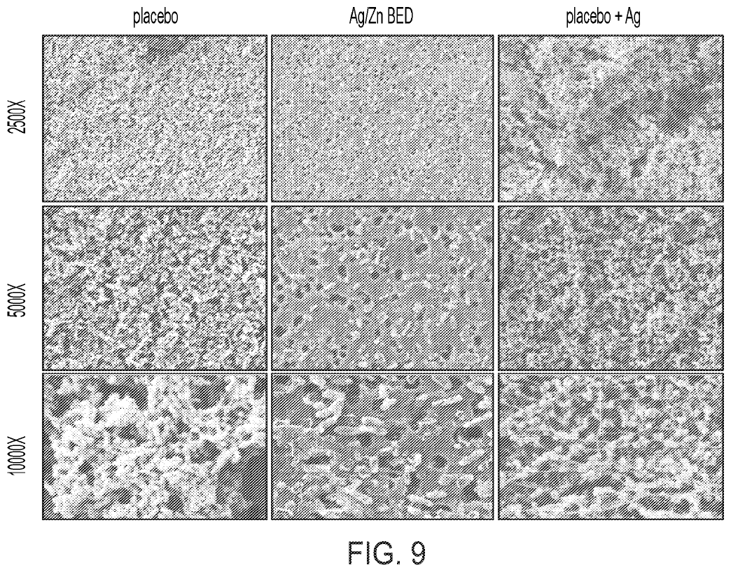

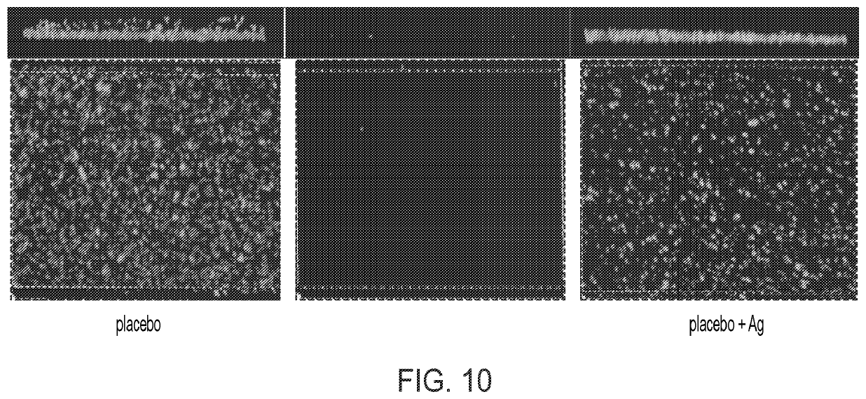

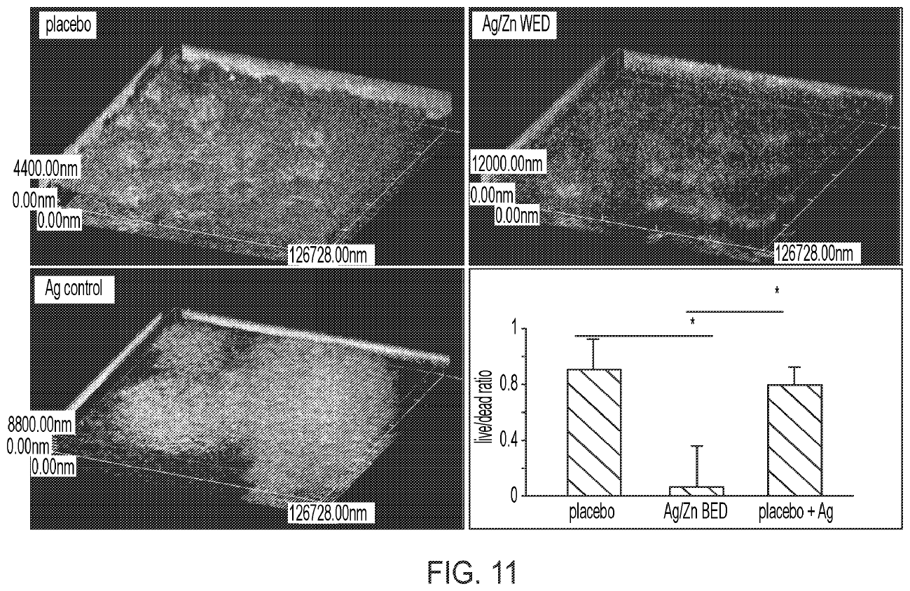

As illustrated in FIG. 8A, P. aeruginosa was grown in round bottom tubes in LB medium with continuous shaking and absorbance was measured by calculating optical density at 600 nm at different time points. It was observed that Ag/Zn BED and the control dressing with equal amount of silver inhibited bacterial growth (n=4) (FIG. 8B,C). When bacteria is grown in an agar plate with Ag/Zn BED dressing or placebo embedded in the agar, the zone of inhibition is clearly visible in the case of Ag/Zn BED thus demonstrating its bacteriostatic property, while placebo with silver dressing showed a smaller zone of inhibition, indicating the effect role of electric field as compared to topical contact. (FIG. 8D). However, as evident from scanning electron microscope images (FIG. 9); EPS staining (FIG. 10); and live/dead staining (FIG. 11), Ag/Zn BED disrupts biofilm much better while silver does not have any effect on biofilm disruption. Silver has been recognized for its antimicrobial properties for centuries. Most studies on the antibacterial efficacy of silver, with particular emphasis on wound healing, have been performed on planktonic bacteria. Silver ions, bind to and react with proteins and enzymes, thereby causing structural changes in the bacterial cell wall and membranes, leading to cellular disintegration and death of the bacterium. Silver also binds to bacterial DNA and RNA, thereby inhibiting the basal life processes.

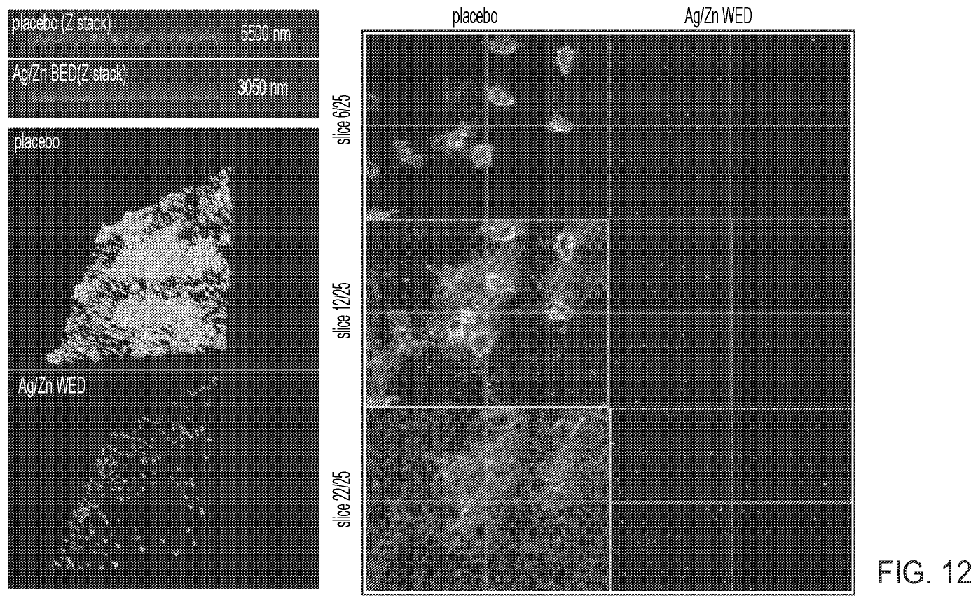

Silver is effective against mature biofilms of P. aeruginosa, but only at a high silver concentration. A concentration of 5-10 μg/mL silver sulfadiazine has been reported to eradicate biofilm whereas a lower concentration (1 μg/mL) had no effect. Therefore, the concentration of silver in currently available wound dressings is much too low for treatment of chronic biofilm wounds. FIG. 12 shows PA01 staining of the biofilm demonstrating the lack of elevated mushroom like structures in the Ag/Zn BED treated sample.

### Aq/Zn BED Down-Regulates Quorum Sensing Genes

The pathogenicity of P. aeruginosa is attributable to an arsenal of virulence factors. The production of many of these extracellular virulence factors occurs only when the bacterial cell density has reached a threshold (quorum). Quorum sensing is controlled primarily by two cell-to-cell signaling systems, called las and rhl, which are both composed of a transcriptional regulator (LasR and RhlR, respectively) and an autoinducer synthase (LasI and RhlI, respectively). In P. aeruginosa, LasI produces 30C12-HSL. LasR, then, responds to this signal and the LasR:30C12-HSL complex activates transcription of many genes including rhIR, which encodes a second quorum sensing receptor, RhlR which binds to autoinducer C4-HSL produced by RhlI. RhlR:C4-HSL also directs a large regulon of genes. P. aeruginosa defective in QS is compromised in their ability to form biofilms. Quorum sensing inhibitors increase the susceptibility of the biofilms to multiple types of antibiotics.

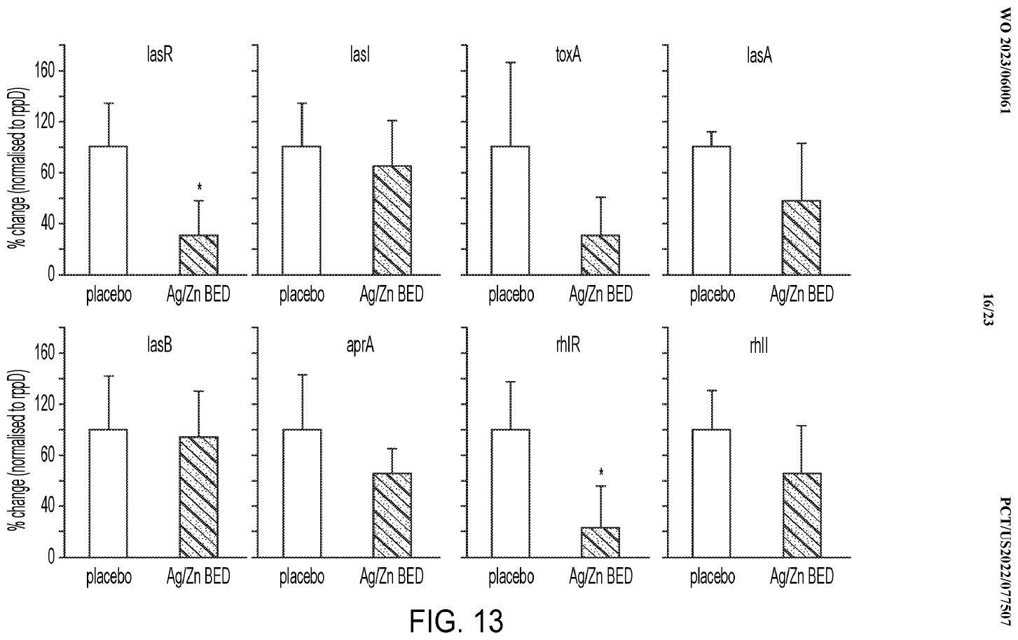

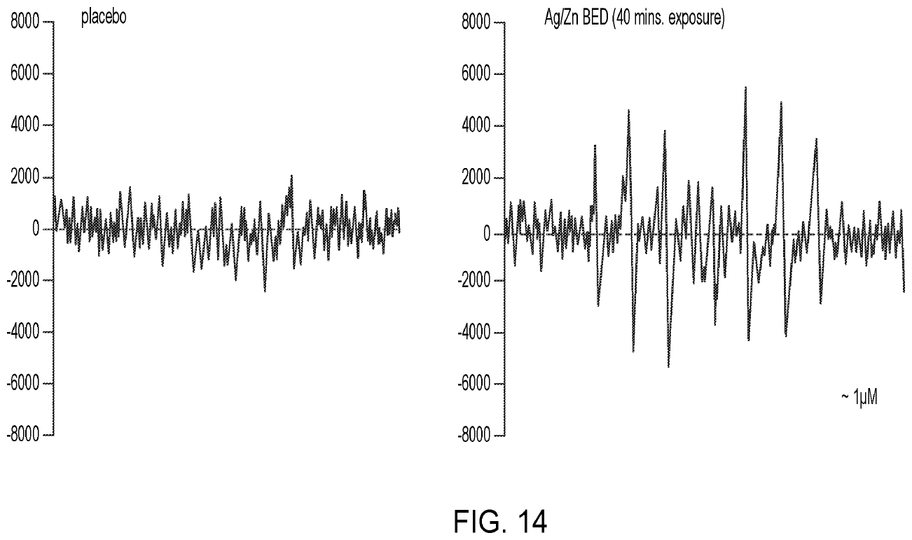

To test the effect of the electric field on quorum sensing genes, we subjected the mature biofilm to the Ag/Zen BED or placebo for 12 hours and looked at gene expression levels. We selected an earlier time point, because by 24 hours, as in earlier experiments, most bacteria under Ag/Zn BED treatment were dead. We found a significant down regulation of lasR and rhIR (n=4, p<0.05). lasR transcription has been reported to weakly correlate with the transcription of lasA, lasB, toxA and aprA. We did not, however, find any significant difference in their expression levels at this time point, although we found them down regulated in the Ag/Zn BED treated samples at the 24 hour time point (data not shown). (FIG. 13).

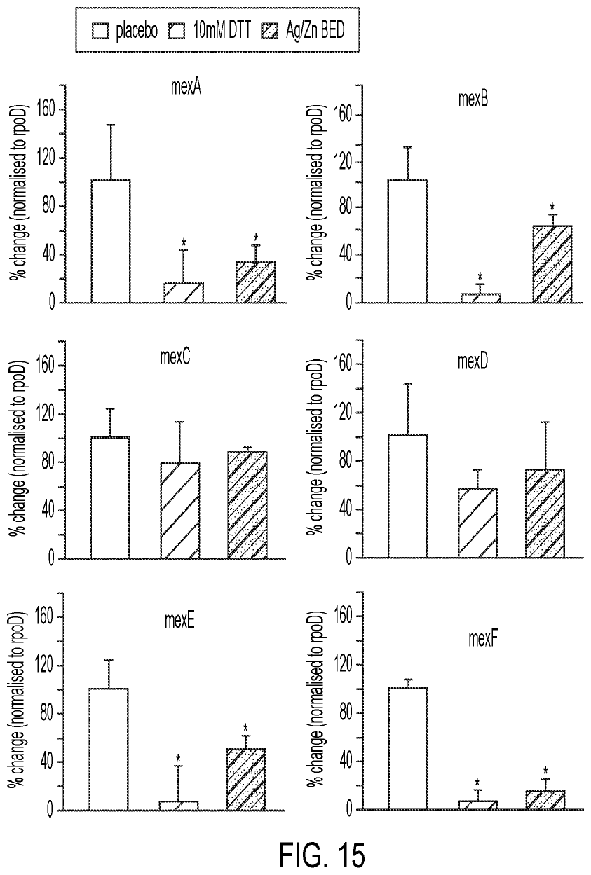

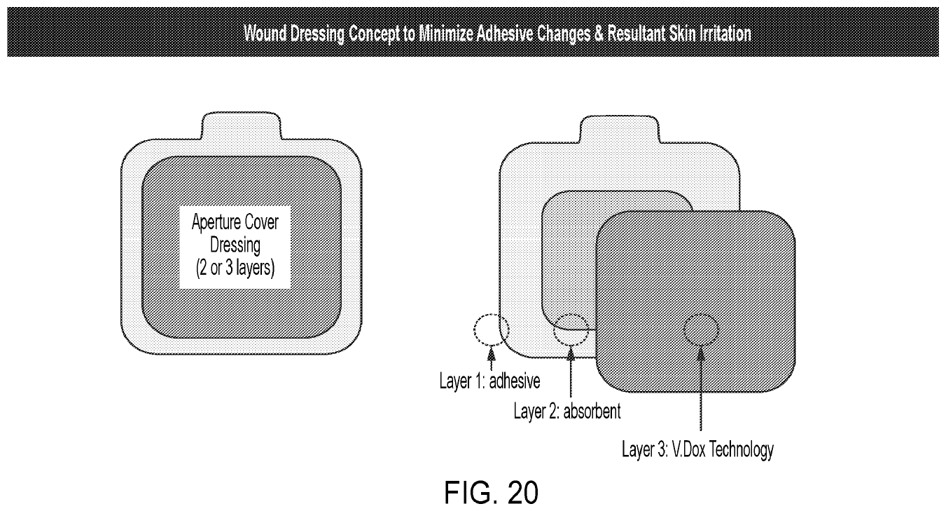

Aq/Zn BED represses the redox sensing multidrug efflux system in P. aeruginosa Ag/Zn BED acts as a reducing agent and reduces protein thiols. One electron reduction of dioxygen 02, results in the production of superoxide anion. Molecular oxygen (dioxygen) contains two unpaired electrons. The addition of a second electron fills one of its two degenerate molecular orbitals, generating a charged ionic species with single unpaired electrons that exhibit paramagnetism. Superoxide anion, which can act as a biological reductant and can reduce disulfide bonds, is finally converted to hydrogen peroxide is known to have bactericidal properties. Here, we used electron paramagnetic resonance (EPR) to detect superoxide directly upon exposure to the bioelectric dressing. Superoxide spin trap was carried out using DEPMPO (2-(diethoxyphosphoryl)-2-methyl-3,4-dihydro-2H-pyrrole 1-oxide) and ˜1 μM superoxide anion production was detected upon 40 mins of exposure to Ag/Zn BED (FIG. 20). MexR and MexT are two multidrug efflux regulators in P. aeruginosa which uses an oxidation-sensing mechanism. Oxidation of both MexR and MexT results in formation of intermolecular disulfide bonds, which activates them, leading to dissociation from promoter DNA and de-repression of MexAB-oprM and MexEF-oprN respectively, while in a reduced state, they do not transcribe the operons. Induction of Mex operons leads not only to increased antibiotic resistance but also to repression of the quorum sensing cascades and several virulence factors. We observe down-regulation of the downstream Mex genes MexA, MexB, MexE and MexF (but not MexC and MexD) (n=4, p<0.05), in Ag/Zn BED treated samples, inactive forms of MexR and MexT in their reduced states. To confirm the reducing activity of the Ag/Zn BED, the experiments were repeated with 10 mM DTT and similar results were observed. (FIG. 15).

### Ag/Zn BED Diminishes Glycerol-3-Phosphate Dehydrogenase Enzyme Activity

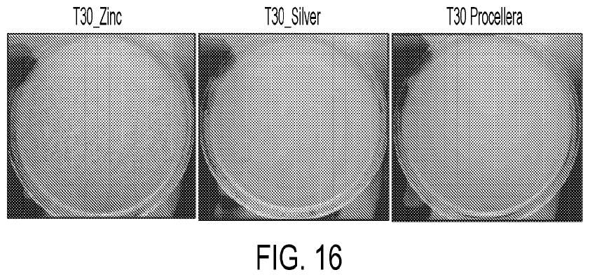

Electric fields can affect molecular charge distributions on many enzymes. Glycerol-3-phosphate dehydrogenase is an enzyme involved in respiration, glycolysis, and phospholipid biosynthesis and is expected to be influenced by external electric fields in P. aeruginosa. We observed significantly diminished glycerol-3-phosphate dehydrogenase enzyme activity by treating P. aeruginosa biofilm to the Ag/Zn BED for 12 hours (n=3). (FIG. 16).

### Example 8

### Viral Proliferation Test

A disclosed embodiment was tested against several viral strains. According to the results, there was 100% kill after a 104 PFU viral challenge/sample.

| Virus Kind | Influenza Virus | Feline Calcivirus |
|---|---|---|
| Virus | Influenza A Virus (H3N2) | Feline calicivirus |
| Strain | Influenza A Virus (H1N1) | Strain: F-9 |
| Host Cell | MCDK Cell (Dog kidney | CRFK Cell (Cat kidney |
| | cell origin) | cell origin) |

| | |
|---|---|
| Time | # of Plaques of Fabrics (Vomaris) |

| | | | | | |
|---|---|---|---|---|---|
| point | Blank | Zinc | Silver | Procellera | Notes |

| | | | | |
|---|---|---|---|---|
| T30 | TMTC | TMTC | 0 | 0 |
| T60 | TMTC | TMTC | 0 | 0 |

TMTC, too many to count plaques

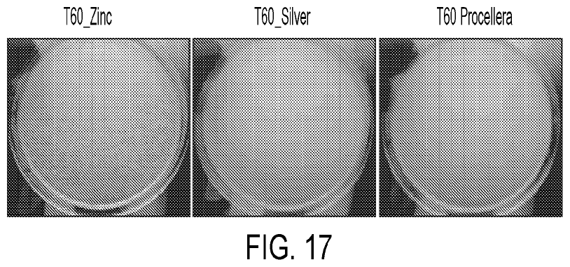

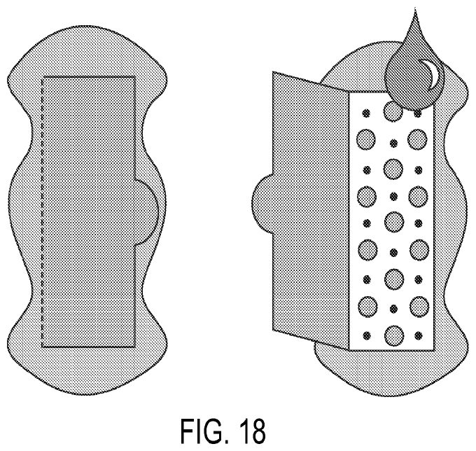

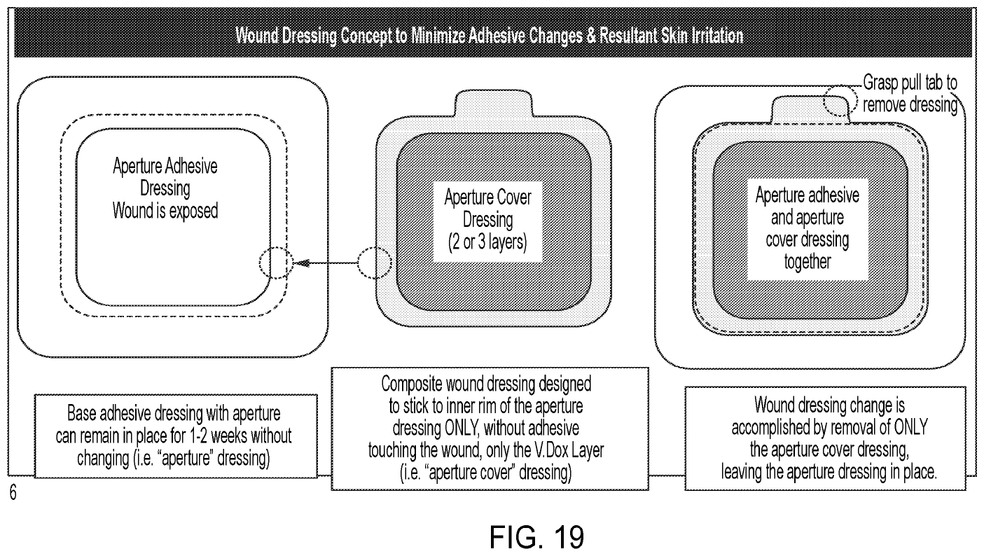

The antiviral effects were observed within minutes of exposure, as shown in FIG. 16 and FIG. 17.

### Example 9

### Treatment of Hidradenitis Suppurativa

A 35-year old male suffers from hidradenitis suppurativa. A disclosed embodiment is applied to the treatment area. The cover substrate comprising a reversibly attachable window is left on the skin for 5 days, while the absorbent substrate is changed several times a day by opening the window to access the substrate.

### Example 10

### Treatment of Hidradenitis Suppurativa

A 55-year old female suffers from hidradenitis suppurativa. A disclosed embodiment is applied to the treatment area. The cover substrate comprising a reversibly attachable window is left on the skin for 5 days, while the absorbent substrate is changed several times a day by opening the window to access the substrate. The substrate comprises electrodes that establish a LLEC in the treatment area.

### Example 11

### Skin Assessment

A 25-year old male suffering from a MARSI has his skin assessed for:
a. Skin temperature;
b. Skin color;
c. Skin moisture level;
d. Skin turgor (fullness and elasticity);
e. Skin fragility; and
f. Skin integrity.

Based on the assessment, the patient is treated with a disclosed embodiment.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present disclosure, which is defined solely by the claims. Accordingly, embodiments of the present disclosure are not limited to those precisely as shown and described.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present disclosure, which is defined solely by the claims. Accordingly, embodiments of the present disclosure are not limited to those precisely as shown and described.

Certain embodiments are described herein, comprising the best mode known to the inventor for carrying out the methods and devices described herein. Of course, variations on these described embodiments will become apparent to those of ordinary skill in the art upon reading the foregoing description. Accordingly, this disclosure comprises all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

Groupings of alternative embodiments, elements, or steps of the present disclosure are not to be construed as limitations. Each group member may be referred to and claimed individually or in any combination with other group members disclosed herein. It is anticipated that one or more members of a group may be comprised in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the disclosure are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope otherwise claimed. No language in the present specification should be construed as indicating any non-claimed element essential to the practice of embodiments disclosed herein.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present disclosure so claimed are inherently or expressly described and enabled herein.

1. A wound management system comprising; an absorbent substrate; and a cover substrate shaped to fit over said absorbent substrate, said cover substrate further comprising a perimeter comprising an adhesive, wherein said substrate further comprises at least one slit forming a flap within the perimeter sufficient to allow access to the absorbent substrate.

2. The wound management system of claim 1, wherein said absorbent substrate comprises two or more biocompatible electrodes configured to generate at least one of a low level electric field (LLEF) or low level electric current (LLEC).

3. The wound management system of claim 2, wherein at least one electrode is silver and at least one electrode is zinc.

4. The wound management system of claim 1, wherein said at least one slit comprises two substantially parallel slits that are joined at their ends by a third slit.

5. The wound management system of claim 4, wherein said third slit joins the two substantially parallel slits at about a 90 degree angle.

6. The wound management system of claim 4, wherein said cover substrate is clear.

7. The wound management system of claim 1, further comprising a substrate comprising two or more biocompatible electrodes configured to generate at least one of a low level electric field (LLEF) or low level electric current (LLEC).

8. The wound management system of claim 7, wherein at least one electrode is silver and at least one electrode is zinc.

9. The wound management system of claim 8, wherein said at least one slit comprises two substantially parallel slits that are joined at their ends by a third slit.

10. The wound management system of claim 9, wherein said third slit joins the two substantially parallel slits at about a 90 degree angle.

11. The wound management system of claim 9, wherein said cover substrate is clear.

12. A method of dressing a wound, comprising applying to the wound the management system of claim 1.

13. The method of claim 12, wherein said wound comprises a surgical incision.

14. The method of claim 13, wherein said surgical incision comprises a dental surgical incision.

15. A method of dressing a wound, comprising applying to the wound the management system of claim 7.
